(19) United States
(12) Patent Application Publication
(10) Pub. No.: US 2016/0374645 A1
(43) Pub. Date: Dec. 29, 2016

(54) METHOD FOR PERFORMING LOW POWER MODE IN PORTABLE ULTRASONIC DIAGNOSTIC APPARATUS AND PORTABLE ULTRASONIC DIAGNOSTIC APPARATUS FOR APPLYING SAME

(71) Applicants: DONGGUK UNIVERSITY INDUSTRY-ACADEMIC, Seoul (KR); HEALCERION CO., LTD., Seoul (KR)

(72) Inventors: Sung Min KIM, Gyeonggi-do (KR); Jeong Won RYU, Seoul (KR); Jae Hoon JEONG, Seoul (KR)

(21) Appl. No.: 15/101,622

(22) PCT Filed: Dec. 5, 2014
(86) PCT No.: PCT/KR2014/011902

§ 371 Date: Jun. 3, 2016

(30) Foreign Application Priority Data
Dec. 5, 2013 (KR) 10-2013-0150368

(51) Int. Cl. A61B 8/00; A61B 8/14; A61B 8/08

(52) U.S. Cl. CPC A61B 8/4427; A61B 8/54; A61B 8/565; A61B 8/0866; A61B 8/145; A61B 8/5207; A61B 8/5223; A61B 8/4444; A61B 8/4494; A61B 8/465

(57) ABSTRACT

Provided are a method of performing a low power mode of a portable ultrasonic diagnostic apparatus and a portable ultrasonic diagnostic apparatus for applying the same. The method include stopping a circuit operation related to a receiving circuit which receives an ultrasonic signal reflected by a test subject when the ultrasonic signal is transmitted to obtain an ultrasonic image of the test subject and stopping a circuit operation of a transmitting circuit which transmits the ultrasonic signal when the ultrasonic signal reflected by the test subject is received. Accordingly, power consumed by the portable ultrasonic diagnostic apparatus may be minimized.

### FIELD

The present invention relates to a method of performing a low power mode of a portable ultrasonic diagnostic apparatus and a portable ultrasonic diagnostic apparatus for using the same, and more particularly, to a method of performing a low power mode of a portable ultrasonic diagnostic apparatus using a battery with limited power as a power source and a potable ultrasonic diagnostic apparatus for using the same.

### BACKGROUND

With noninvasive and nondestructive properties, ultrasonic diagnostic apparatuses are generally used in the medical field to obtain information of the inside of an object. Since a high-resolution image of internal organizations of the object may be provided to a doctor with no surgical operations of directly incising and observing the object, ultrasonic diagnostic systems are very importantly used in the medical field.

Ultrasonic diagnostic apparatuses are systems which emit an ultrasonic signal from a body surface of a test subject toward a target portion inside the test subject, extract information from a reflected ultrasonic signal, and obtain an image of a section of soft tissue or a blood flow in a noninvasive mode.

Compared with other imaging diagnostic apparatuses such as X-ray inspection apparatuses, computerized tomography (CT) scanners, magnetic resonance image (MRI) scanners, and nuclear medicine inspection apparatuses, ultrasonic diagnostic systems described above have a small size, are cheap, may display in real time, and have excellent safety without being exposed to X-rays, thereby being generally used to diagnose hearts, internal organs in an abdominal cavity, urinary systems, and genital organs.

Ultrasonic diagnostic apparatuses each include a switching portion which forms transmitting and receiving paths for transmitting an ultrasonic signal to the test subject and receiving the ultrasonic signal reflected by the test subject to obtain an ultrasonic image of the test subject.

Since conventional ultrasonic diagnostic apparatuses use an alternating current (AC) power source which supplies power constantly, a lack of power does not occur. Recently, as portable ultrasonic diagnostic apparatuses using a battery with limited power as a power source have been used, interest in a technology of providing a maximal use time with minimal power has increased.

As a prior art document related to the present invention, there is Korean Patent Publication No. 10-2010-0050845 (published on May 14, 2010).

### DISCLOSURE

### Technical Problem

An aspect of the present invention is to provide a method of performing a low power mode of a portable ultrasonic diagnostic apparatus using a battery with limited power as a power source to minimize power consumed by the portable ultrasonic diagnostic apparatus and a portable ultrasonic diagnostic apparatus for using the same.

### Technical Solution

One aspect of the present invention provides a method of performing a low power mode of a portable ultrasonic diagnostic apparatus which includes a TX circuit for applying power to a high voltage pulse generator generating an electric pulse to generate an ultrasonic wave to be transmitted to a test subject, an RX circuit for applying power to an analog-digital (AD) signal processor amplifying an ultrasonic echo signal returning from the test subject and then converting the amplified ultrasonic echo signal into a digital signal, and an external input terminal for controlling low power modes of the power applied to the TX circuit and the RX circuit. The method includes applying operating power to the TX circuit before a wake-up time ΔT1 necessary for driving the TX circuit to transmit an ultrasonic signal, entering, by the RX circuit for receiving the ultrasonic echo signal, a low power mode (or a power-off state); applying, by an ultrasonic probe, the electric pulse to a piezoelectric element array module to generate the ultrasonic wave to obtain an ultrasonic image of the test subject, receiving, by a menu input portion, whether to set a standby time σT of the RX circuit for receiving the ultrasonic echo signal corresponding to an area at a particular depth according to a user selection, applying operating power to the RX circuit and then allowing the TX circuit to be in a low power mode (or a power-off state) when the standby time σT of the RX circuit is not set and allowing the TX circuit to be in the low power mode (or the power-off state) and then applying the operating power to the RX circuit when the standby time σT is set, and receiving and analyzing, by a main circuit portion of the ultrasonic diagnostic apparatus, the echo signal to generate and transmit the ultrasonic image to a user screen.

The applying operating power to the RX circuit and then allowing the TX circuit to be in the low power mode (or the power-off state) when the standby time σT of the RX circuit is not set and allowing the TX circuit to be in the low power mode (or the power-off state) and then applying the operating power to the RX circuit when the standby time σT is set may include applying the operation power to the RX circuit before a wake-up time ΔT2 necessary for driving the RX circuit to receive the ultrasonic echo signal and allowing the TX circuit for transmitting the ultrasonic signal to be in the low power mode (or the power-off state) when the standby time σT is not set (σT=0) and allowing the TX circuit for transmitting the ultrasonic signal to be in the low power mode (or the power-off state), standing by while additionally applying the standby time σT to the wake-up time ΔT2 necessary for driving the RX circuit to receive the ultrasonic echo signal, and applying the operating power to the RX circuit when the standby time σT is set.

Another aspect of the present invention provides a portable ultrasonic diagnostic apparatus, to which the method of performing the low power mode according to claims 1 and 2 is to be applied, including an ultrasonic probe which includes a piezoelectric element array module and a multiplexer (MUX) circuit portion to generate an ultrasonic wave and receive an echo signal, a main circuit portion which receives and analyzes the echo signal received from the ultrasonic probe to generate and transmit an ultrasonic image to a user screen, a portable battery which supplies power necessary for the ultrasonic probe and the main circuit portion, and a low power mode controller which receives power from the portable battery to have a high voltage which drives the ultrasonic probe and generates and distributes a voltage necessary for the entire system.

Here, the low power mode controller may allow the high voltage pulse generator to operate on a preset frequency in an operation time of the TX circuit for receiving the voltage from the battery and transmitting an ultrasonic pulse and allows the AD signal processor provided in the main circuit portion to amplify and then convert the ultrasonic echo signal into the digital signal in an operation time of the RX circuit which receives the ultrasonic echo.

Here, the main circuit portion may include a transceiver which performs as a switch connecting one of a TX circuit for transmitting the ultrasonic wave and an RX circuit for receiving an ultrasonic echo to the ultrasonic probe depending on a transmitting and receiving state, and the low power mode controller, by controlling the transceiver, may minimize a power consumption amount by stopping an operation of the RX circuit which receives an ultrasonic echo signal reflected by a test subject when the ultrasonic signal is transmitted and stopping an operation of the TX circuit which transmits the ultrasonic signal when the ultrasonic echo signal is received.

Also, the main circuit portion may include a high voltage generator which generates an electric pulse applied to the piezoelectric element array module to generate the ultrasonic wave, an AD signal processor which amplifies a level of the ultrasonic echo signal returning from the test subject and converts the amplified ultrasonic echo signal into a digital signal, the transceiver which transmits a high voltage pulse generated by the high voltage pulse generator to the ultrasonic probe and transmits an analog signal received from the ultrasonic probe to the AD signal processor, a beam former which allows the high voltage pulse generator to generate an adequate high voltage pulse using a parameter adequate to the ultrasonic probe and receives the digital signal from the AD signal processor to perform data conversion to be appropriate for the ultrasonic probe, a processor which allows the beam former to perform beam forming adequate to the ultrasonic probe, generates the ultrasonic image using data received from the beam former, transmits the ultrasonic image to a display portion and an external display apparatus using ultrasonic scan data, and controls the entire system, and a communication portion which transmits and receives data with the external display apparatus.

Also, the communication portion may use any one of a local area network (LAN) using a cable, Bluetooth, a wireless universal serial bus (USB), a wireless LAN, wireless fidelity (Wi-Fi), Zigbee, and infrared data association (IrDA).

In addition, the external display apparatus may include a data communication portion which transmits and receives data with the communication portion, a menu input portion which receives a menu signal from a user, a screen display portion which displays the ultrasonic image and a menu, and a controller which transmits and receives a control signal with the processor.

Meanwhile, the data communication portion may receive scan data from the portable ultrasonic diagnostic apparatus and may transmit the scan data to the controller, the controller may perform a scan conversion process of forming the ultrasonic image using the scan data and then may perform post processing necessary for improving image quality, the controller may perform a decompression process when the scan data sent from the portable ultrasonic diagnostic apparatus is compressed, the screen display portion may display the ultrasonic image formed by the controller on a screen to allow the user to see it, the menu input portion may receive and transmit a user input to the controller, and the controller may directly process the user input or may transmit the user input to the portable ultrasonic diagnostic apparatus using the data communication portion.

### Advantageous Effects

As described above, a method of performing a low power mode in which a circuit operation related to a receiving circuit which receives an ultrasonic echo signal reflected by a test subject is stopped when an ultrasonic signal is transmitted to obtain an ultrasonic image of the test subject and a circuit operation related to a transmitting circuit which transmits the ultrasonic signal is stopped when the ultrasonic echo signal reflected by the test subject is received is provided, thereby reducing power consumed by a portable ultrasonic diagnostic apparatus to be minimized.

Also, according to the present invention, the power consumed by the portable ultrasonic diagnostic apparatus which operates in the low power mode may be additionally reduced using a wake-up time ΔT1 related to the transmitting circuit which transmits the ultrasonic signal to obtain the ultrasonic image of the test subject, a wake-up time ΔT2 related to the receiving circuit which receives the ultrasonic echo signal reflected by the test subject, and a standby time σT of standing by to obtain a necessary area of the ultrasonic image of the test subject except an unnecessary particular area.

In addition, according to the present invention, it is possible to provide a portable ultrasonic diagnostic apparatus which includes a low power mode controller allowing the portable ultrasonic diagnostic apparatus to operate in a low power mode using minimal power by controlling a switching portion forming transmitting and receiving paths to perform operations of transmitting an ultrasonic signal to a test subject to obtain an ultrasonic image of the test subject and receiving an ultrasonic echo signal reflected by the test subject and operates in a low power mode.

### BRIEF DESCRIPTION OF DRAWINGS

FIG. 1 is a configuration diagram of an ultrasonic diagnostic apparatus for using a method of performing a low power mode of a portable ultrasonic diagnostic apparatus according to one embodiment of the present invention.

FIG. 2 is a view illustrating a detailed configuration of a main circuit portion of FIG. 1.

FIG. 3 is a schematic configuration diagram of an external display apparatus connected to the portable ultrasonic diagnostic apparatus according to one embodiment of the present invention.

FIG. 4 is a view illustrating operation times of a TX circuit and an RX circuit which operate when the portable ultrasonic diagnostic apparatus to which the embodiment of the present invention is applied transmits an ultrasonic pulse and receives an ultrasonic echo.

FIGS. 5 and 6 are views illustrating detailed configurations related to the operation times of the TX circuit and the RX circuit shown in FIG. 4.

FIG. 7 is a view illustrating a time of actually applying power obtained by applying a wake-up time ΔT1 necessary for driving the TX circuit and a wake-up time ΔT2 necessary for driving the RX circuit to the operation times of the TX circuit and the RX circuit shown in FIG. 4.

FIG. 8 is a view illustrating an application of a standby time σT to the operation time of the TX circuit shown in FIG. 7.

FIG. 9 is a view illustrating a schematic comparison between a general ultrasonic image of a fetus and an ultrasonic image of the fetus received by the portable ultrasonic diagnostic apparatus, to which the standby time σT shown in FIG. 8 is applied.

FIG. 10 is a flowchart illustrating the method of performing the low power mode of the portable ultrasonic diagnostic apparatus according to one embodiment of the present invention.

### MODE FOR INVENTION

Hereinafter, exemplary embodiments of the present invention will be described in detail with reference to the attached drawings.

The embodiments of the present invention are provided to more completely explain the present invention to one of ordinary skill in the art. The following embodiments may be modified into various other forms, and the scope of the present invention will not be limited thereto. The embodiments are provided to allow the present disclosure to be more substantial and complete and to fully transfer the inventive concept to those skilled in the art.

The terms are used herein to describe particular embodiments but will not limit the present invention. As used herein, singular expressions, unless defined otherwise in contexts, include plural expressions. Also, it will be understood that the terms “comprises” and/or “comprising” used herein specify the presence of stated shapes, numbers, operations, members, elements, and/or groups thereof, but do not preclude the presence or addition of one or more other shapes, numbers, operations, members, elements, and/or groups thereof. As used herein, the term “and/or” includes any and all combinations of one or more of the associated listed items.

Throughout the specification, although the terms “first”, “second”, etc. may be used herein to describe various members, components, areas, layers, and/or portions, these members, components, areas, layers and/or portions should not be limited by these terms. The terms do not mean a particular order, top and bottom, or merits and demerits but are used only to distinguish one member, area, or portion from others. Accordingly, a first member, area, or portion which will be described below may indicate a second member, area, or portion without deviating from teachings of the present invention.

Hereinafter, the embodiments of the present invention will be described with reference to schematic drawings thereof. Throughout the drawings, for example, according to manufacturing technologies and/or tolerances, modifications of illustrated shapes may be conceived. Accordingly, the embodiments of the present invention will not be understood to be limited to particular shapes of illustrated areas but will include changes in shape caused while being manufactured.

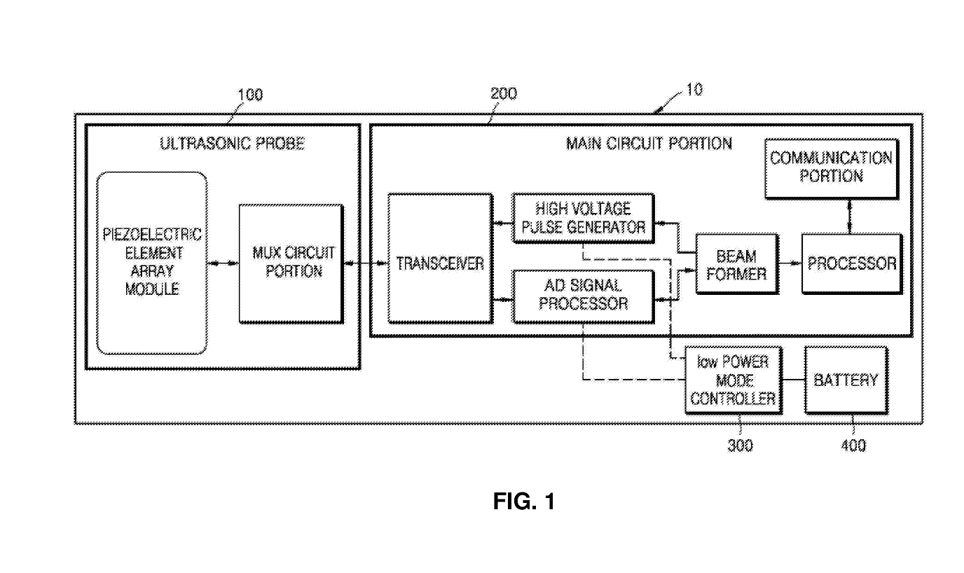

FIG. 1 is a configuration diagram of an ultrasonic diagnostic apparatus for using a method of performing a low power mode of a portable ultrasonic diagnostic apparatus according to one embodiment of the present invention.

Referring to FIG. 1, an ultrasonic diagnostic apparatus 10 according to one embodiment of the present invention includes an ultrasonic probe 100, a main circuit portion 200, a low power mode controller 300, and a battery 400.

First, the ultrasonic probe 100 includes a piezoelectric element array module 110 and a multiplexer (MUX) circuit portion 120. Here, the piezoelectric element array module 110 includes a piezoelectric element and generates an ultrasonic wave and the MUX circuit portion 120 receives an echo signal. The main circuit portion 200 forms an ultrasonic image by receiving and analyzing the echo signal and transmits the ultrasonic image to an external portable display apparatus 500 which has a user screen. Also, the low power mode controller 300 includes a high voltage which drives the ultrasonic probe 100 to generate and distribute a voltage necessary for the entire system and minimizes a power consumption amount during an operation to provide a maximal use time with the battery 400 with limited power as a power source.

In detail, the piezoelectric element array module 110 is formed of a piezoelectric material. The piezoelectric material may perform two functions of oscillating to generate and transmit a pulse of a sound wave into a human body or receiving and transducing a reflected echo into an electrical signal. Recently, as the piezoelectric material, piezoelectric ceramic such as lead zirconatetitanate (PZT) having highest electro-acoustic conversion efficiency is generally used. The piezoelectric element array module 110 is generally configured to arrange a large number of, such as 64, 128, 192, etc., piezoelectric elements in an array. Here, a range of an electric pulse which drives a piezoelectric element corresponds to a high voltage from +100V to −100V. The piezoelectric element array module 110 is also referred to as an ultrasound transducer.

The MUX circuit portion 120 reduces the number of signal pins. The MUX circuit portion 120 matches signal lines between the piezoelectric element array module 110 and a transceiver 210.

That is, in transmitting of an ultrasonic wave and receiving an echo, since all the elements in the piezoelectric element array module 110 are not used at the same time and only some elements at a position for collecting ultrasonic echo data are used, the elements are selectively electrically connected to the transceiver 210.

As described above, generally, the number of the piezoelectric elements of the piezoelectric element array module 110 may be large such as 64, 128, 192, etc. When the MUX circuit portion 120 is used as described above, the number of signal lines becomes notably reduced.

Meanwhile, the main circuit portion 200 may control to generate an ultrasonic wave to the test subject, may receive an echo signal received from the piezoelectric element array module 110, may analyze a difference in intensity of the echo signals, and process the difference into the brightness of a dot, thereby generating an ultrasonic image.

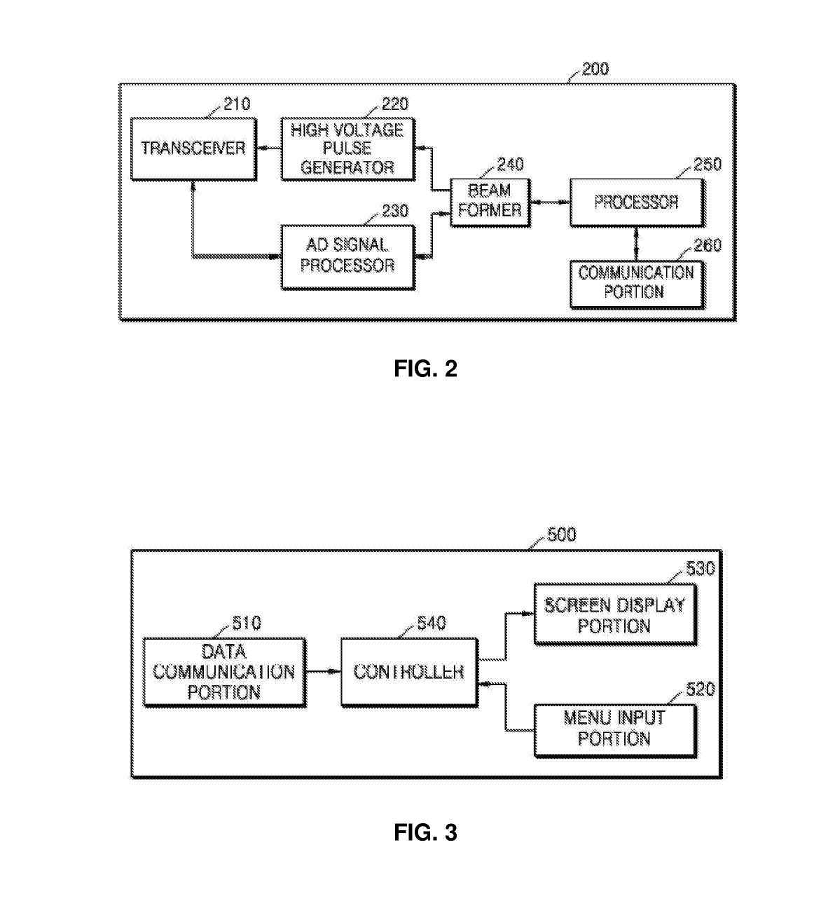

FIG. 2 is a view illustrating a detailed configuration of the main circuit portion of FIG. 1.

As shown in the drawing, the main circuit portion 200 includes the transceiver 210, a high voltage pulse generator 220, an analog digital (AD) signal processor 230, a beam former 240, a processor 250, and a communication portion 260.

The transceiver 210 transmits a high voltage pulse generated by the high voltage pulse generator 220 to the ultrasonic probe 100 or transmits an analog signal received from the ultrasonic probe 100 to the AD signal processor 230. That is, the transceiver 210 is a switch which connects a TX circuit with the piezoelectric element array module 110 to transmit an ultrasonic wave and connects an RX circuit with the piezoelectric element array module 110 to receive an ultrasonic echo.

The high voltage pulse generator 220 generates an electric pulse to be applied to the piezoelectric element array module 110 to generate the ultrasonic wave. The AD signal processor 230 amplifies an ultrasonic echo signal which returns from the test subject with a very small level and coverts the ultrasonic echo signal into a digital signal.

The beam former 240 allows the high voltage pulse generator 220 to generate an adequate high voltage pulse using a parameter adequate to the ultrasonic probe 100, which is referred to as TX beam forming. Here, an electric pulse is delayed with a time according to a position of a piezoelectric element to concentrate energy of an ultrasonic wave on a focus at a particular distance when the ultrasonic wave is transmitted. A digital signal converted by the AD signal processor 230 is received and data-converted according to the ultrasonic probe 100 and then transmitted to the processor 250, which is referred to as RX beam forming. Here, an electrical signal output by each piezoelectric element is delayed with a time according to a position and receiving time of the piezoelectric element when an ultrasonic echo is received and the time-delayed signals are added to generate ultrasonic data (scan data).

Also, the beam former 240 generates and transmits an adequate digital signal to the AD signal processor 230 under the control of the processor 250.

The processor 250 controls the beam former 240 to perform beam forming adequate to the ultrasonic probe 100, generates an ultrasonic image using data received from the beam former 240, transmits ultrasonic scan data to the external display apparatus 500 using the communication portion 260, and controls the entire system. Also, the processor 250 compresses scan data as necessary to reduce a bandwidth of a transmission line used for communication.

The communication portion 260 is a communication module which transmits and receives data with an external electric device. The communication module may use a wired or wireless communication method and may be a module using one of a wired cable such as a universal serial bus (USB) cable, etc., as the wired communication method, Bluetooth, a wireless USB, a wireless local area network (LAN), wireless fidelity (Wi-Fi), Zigbee, and infrared data association (IrDA), as the wireless communication method.

The communication portion 260 may display the generated ultrasonic image on a display portion of the external display apparatus 500 under the control of the processor 250. Here, the external display apparatus 500 may be a personal computer (PC), a tablet type device, a pad type device, personal digital assistants (PDA), etc. As shown in FIG. 3, the external display apparatus 500 may include a data communication portion 510 which transmits and receives data with the communication portion 260, a menu input portion 520 which receives a menu signal input from a user, a screen display portion 530 which displays an ultrasonic image and a menu, and a controller 540 which transmits and receives a control signal with the processor 250. Here, the data communication portion 510 of the external display apparatus 500 receives scan data from the portable ultrasonic diagnostic apparatus and transmits the scan data to the controller 540. The controller 540 performs a scan conversion process of forming an ultrasonic image using the scan data and then performs post processing necessary for improving image quality. The controller 540 performs a decompressing process when the scan data sent from the portable ultrasonic diagnostic apparatus are compressed. Also, the screen display portion 530 displays the ultrasonic image formed by the controller 540 on a screen to allow the user to see it. The menu input portion 520 receives and transmits a user input to the controller 540. The controller 540 directly process the user input or transmits the user input to the portable ultrasonic diagnostic apparatus using the data communication portion 510.

Also, the ultrasonic diagnostic apparatus 10 according to the embodiment of the present invention may include a display portion (not shown) autonomously. That is, the ultrasonic diagnostic apparatus 10 may transmit the generated ultrasonic image to another electronic device through the communication module to display or may be configured to directly display on the display portion thereof.

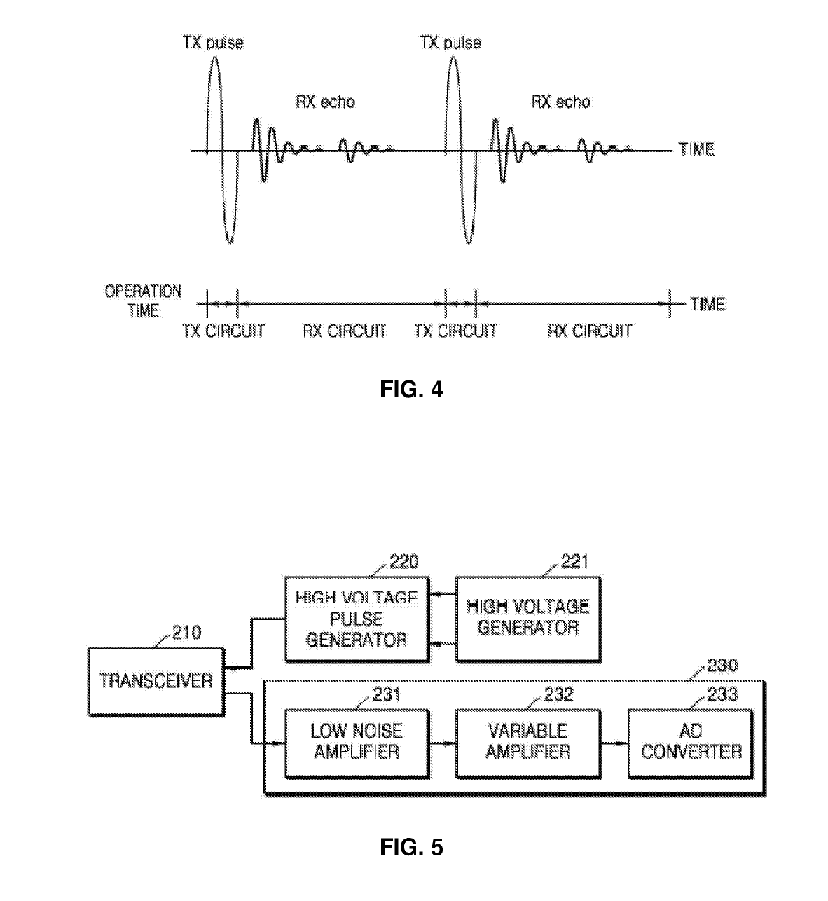

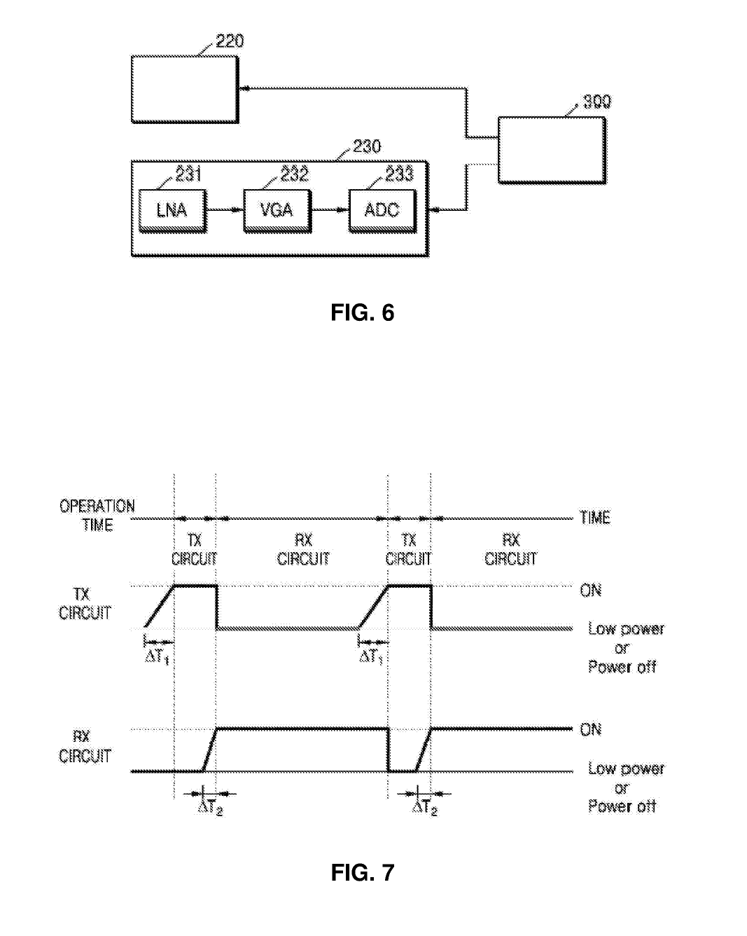

FIG. 4 is a view illustrating operation times of a TX circuit and an RX circuit which operate when the portable ultrasonic diagnostic apparatus to which the embodiment of the present invention is applied transmits an ultrasonic pulse and receives an ultrasonic echo. FIGS. 5 and 6 are views illustrating detailed configurations related to the operation times of the TX circuit and the RX circuit shown in FIG. 4.

In the portable ultrasonic diagnostic apparatus according to the embodiment of the present invention, due to the low power mode controller 300, during an operation time of the TX circuit which transmits an ultrasonic pulse, the high voltage pulse generator 220 receives a voltage from the battery 400 and operates on a preset frequency. During an operation time of the RX circuit which receives an ultrasonic echo, the AD signal processor 230 amplifies and converts an ultrasonic echo signal which returns from an object into a digital signal.

That is, while the ultrasonic pulse is transmitted, due to the low power mode controller 300, the high voltage pulse generator 220, a high voltage generator 221 which applies a high voltage to the high voltage pulse generator 220, and the AD signal processor 230, which relate to the TX circuit, operate and the RX circuit does not operate.

Also, while the ultrasonic echo is received, due to the low power mode controller 300, an analog front end (not shown) and the AD signal processor 230 including a low noise amplifier 231 which amplifies an RX echo signal with a low power level, a variable amplifier 232 for compensating a decreased signal which returns from a place deep inside a human body, a continuous wave Doppler (CWD: not shown), and an AD converter 233 which converts an analog signal into a digital signal to allow the beam former 240 to process a digital signal, which relate to the RX circuit, operate and the TX circuit does not operate.

Meanwhile, components related to the TX circuit and the RX circuit may be provided as single semiconductor chips or an integrated chip. An external input terminal for controlling a low power mode function according to the embodiment of the present invention may be included. It is possible to allow the low power mode controller 300 to control a low power mode and a normal mode using the external input terminal.

FIG. 7 is a view illustrating a time of actually applying power obtained by applying a wake-up time ΔT1 necessary for driving the TX circuit and a wake-up time ΔT2 necessary for driving the RX circuit to the operation times of the TX circuit and the RX circuit shown in FIG. 4.

As shown in the drawing, the ultrasonic diagnostic apparatus 10 according to the embodiment of the present invention stops a circuit operation related to a receiving circuit which receives an ultrasonic signal reflected by the test subject when an ultrasonic signal is transmitted to obtain an ultrasonic image of the test subject and stops a circuit operation related to a transmitting circuit which transmits an ultrasonic signal when the ultrasonic signal reflected by the test subject is received.

That is, the TX circuit of the ultrasonic diagnostic apparatus 10 stands by in a low power mode (or a power-off state) due to the low power mode controller 300 when the ultrasonic diagnostic apparatus 10 operates to receive an ultrasonic echo signal. Here, a wake-up time for entering a normal operation state to transmit an ultrasonic signal from the power-off state or the low power mode is necessary. In this case, a wake-up time necessary for driving the TX circuit is referred to as ΔT1. Since power is applied at the wake-up time ΔT1 before a normal operation time, the TX circuit is allowed to operate a normal operation. After that, the operation time is finished, the TX circuit immediately enters the low power mode or the power-off state.

Also, the RX circuit of the ultrasonic diagnostic apparatus 10 stands by in a low power mode (or a power off state) due to the low power mode controller 300 when the ultrasonic diagnostic apparatus 10 operates to transmit an ultrasonic signal. Here, a wake-up time for entering a normal operation state to receive an ultrasonic echo signal from the power-off state or the low power mode is necessary. In this case, a wake-up time necessary for driving the RX circuit is referred to as ΔT2. Since power is applied at the wake-up time ΔT2 before a normal operation time, the RX circuit is allowed to operate a normal operation. After that, the operation time is finished, the RX circuit immediately enters the low power mode or the power-off state.

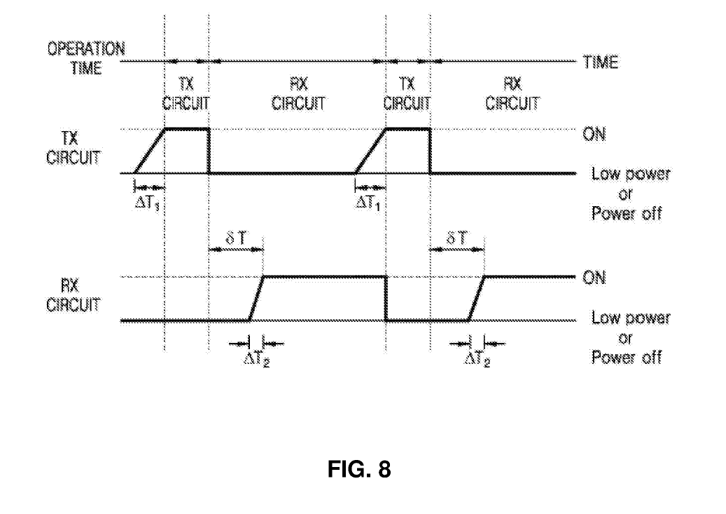

FIG. 8 is a view illustrating an application of a standby time σT to the operation time of the TX circuit shown in FIG. 7.

An area of an ultrasonic image measured by the ultrasonic diagnostic apparatus, in which a medical examiner is interested, is a place positioned of 3 cm or more under a skin of a human body. Particularly, an area present 1-2 cm under the skin of the human body is formed of mostly subcutaneous fat and generally does not have basic and significant information necessary for giving a clinical diagnosis.

Considering the described above, since the portable ultrasonic diagnostic apparatus according to the embodiment of the present invention additionally removes a time for applying power for measuring information of a meaningless area, that is, does not receive an ultrasonic echo signal for a time of reaching an area of a particular depth from the skin of the human body, an unnecessary particular area is excluded from ultrasonic waves of the test subject, thereby additionally reducing power consumed in an operation time corresponding thereto.

As shown in the drawing, the portable ultrasonic diagnostic apparatus according to the embodiment of the present invention receives the ultrasonic echo signal excluding an area of a particular depth input by the user.

Here, the standby time σT refers to a standby time for standing by to obtain a necessary area in the ultrasonic image of the test subject except the unnecessary particular area. Since a transmission speed of ultrasonic waves in a human body is determined to be 1540 m/s, The standby time σT may be accurately calculated by the low power mode controller 300 according to the embodiment of the present invention.

As described above, in the portable ultrasonic diagnostic apparatus according to the embodiment of the present invention, since the RX circuit additionally stands by in the low power mode or the power-off state for the standby time σT and enters a mode for performing a normal operation, power consumed for operating for the standby time σT may be additionally reduced.

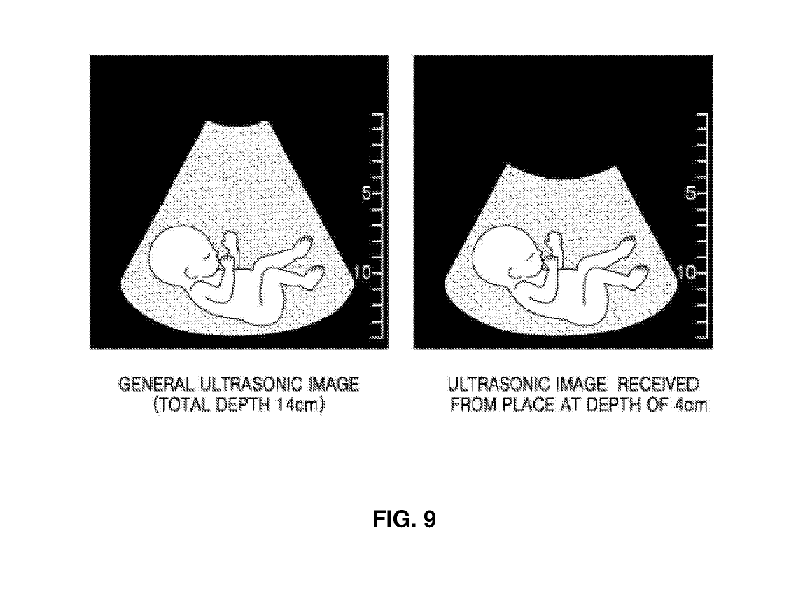

FIG. 9 is a view illustrating a schematic comparison between a general ultrasonic image of a fetus and an ultrasonic image of the fetus received by the portable ultrasonic diagnostic apparatus, to which the standby time σT shown in FIG. 8 is applied.

As shown in the drawing, it may be known that a total depth of the ultrasonic image is 14 cm considering the general ultrasonic image. When it is determined not to receive an ultrasonic echo signal from an area at a depth of 4 cm among them, it is possible to save a part of power necessary for operating the RX circuit to receive the ultrasonic echo signal.

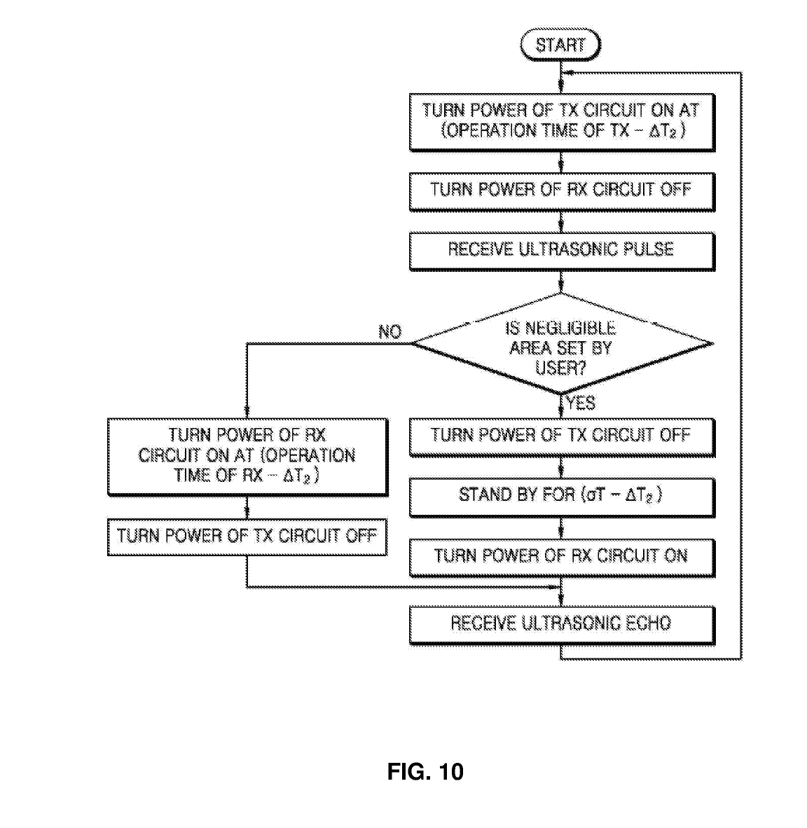

FIG. 10 is a flowchart illustrating the method of performing the low power mode of the portable ultrasonic diagnostic apparatus according to one embodiment of the present invention.

As shown in the drawing, the ultrasonic diagnostic apparatus according to one embodiment of the present embodiment, first, applies operating power to the TX circuit before the wake-up time ΔT1 necessary for driving the TX circuit to transmit an ultrasonic signal (S1). Here, the RX circuit for receiving an ultrasonic echo signal becomes a low power mode (or a power-off state) (S2).

Next, to obtain an ultrasonic image of a test subject, the ultrasonic probe 100 generates an ultrasonic wave by applying an electric pulse to the piezoelectric elements (S3).

Also, the menu input portion 520 receives whether to set the standby time σT of the RX circuit corresponding to an area at a particular depth according to a user selection (S4).

Here, when the standby time σT of the RX circuit is not set (σT=0), operation power is applied to the RX circuit before the wake-up time ΔT2 necessary for driving the RX circuit to receive an ultrasonic echo signal (S5), the TX circuit for transmitting the ultrasonic signal becomes a low power mode (or a power-off state) (S6), and the main circuit portion 200 generates an ultrasonic image by receiving and analyzing the echo signal and transmits the ultrasonic image to a user screen (S10).

On the other hand, when the standby time σT of the RX circuit is set, first, the TX circuit for transmitting the ultrasonic signal becomes the low power mode (a power-off state) (S7).

Next, the standby time σT is additionally applied to the wake-up time ΔT2 necessary for driving the RX circuit to receive the ultrasonic echo signal to stand by (S8), and after that, the operating power is applied to the RX circuit (S9).

Finally, and the main circuit portion 200 generates an ultrasonic image by receiving and analyzing the echo signal and transmits the ultrasonic image to a user screen (S10).

As described above, a method of performing a low power mode in which a circuit operation related to a receiving circuit which receives an ultrasonic echo signal reflected by a test subject is stopped when an ultrasonic signal is transmitted to obtain an ultrasonic image of the test subject and a circuit operation related to transmitting circuit which transmits the ultrasonic signal is stopped when the ultrasonic echo signal reflected by the test subject is received is provided, thereby reducing power consumed by a portable ultrasonic diagnostic apparatus to be minimized.

Also, according to the present invention, the power consumed by the portable ultrasonic diagnostic apparatus which operates in the low power mode may be additionally reduced using a wake-up time ΔT1 related to the transmitting circuit which transmits the ultrasonic signal to obtain the ultrasonic image of the test subject, a wake-up time ΔT2 related to the receiving circuit which receives the ultrasonic echo signal reflected by the test subject, and a standby time σT of standing by to obtain a necessary area of the ultrasonic image of the test subject except an unnecessary particular area.

In addition, according to the present invention, it is possible to provide a portable ultrasonic diagnostic apparatus which includes a low power mode controller allowing the portable ultrasonic diagnostic apparatus to operate in a low power mode using minimal power by controlling a switching portion forming transmitting and receiving paths to perform operations of transmitting an ultrasonic signal to a test subject to obtain an ultrasonic image of the test subject and receiving an ultrasonic echo signal reflected by the test subject and operates in a low power mode.

While the present invention has been particularly shown and described with reference to exemplary embodiments thereof, it will be understood by those of ordinary skill in the art that various changes in form and details may be made therein without departing from the spirit and scope of the present invention as defined by the following claims.

### INDUSTRIAL APPLICABILITY

The present invention will be applied to the field of manufacturing portable ultrasonic diagnostic apparatuses.

1. A method of performing a low power mode of a portable ultrasonic diagnostic apparatus which comprises a TX circuit for applying power to a high voltage pulse generator generating an electric pulse to generate an ultrasonic wave to be transmitted to a test subject, an RX circuit for applying power to an analog-digital (AD) signal processor amplifying an ultrasonic echo signal returning from the test subject and then converting the amplified ultrasonic echo signal into a digital signal, and an external input terminal for controlling low power modes of the power applied to the TX circuit and the RX circuit, the method comprising:
applying operating power to the TX circuit before a wake-up time ΔT1 necessary for driving the TX circuit to transmit an ultrasonic signal;
entering, by the RX circuit for receiving the ultrasonic echo signal, a low power mode (or a power-off state);applying, by an ultrasonic probe, the electric pulse to a piezoelectric element array module to generate the ultrasonic wave to obtain an ultrasonic image of the test subject;
receiving, by a menu input portion, whether to set a standby time σT of the RX circuit for receiving the ultrasonic echo signal corresponding to an area at a particular depth according to a user selection;
applying operating power to the RX circuit and then allowing the TX circuit to be in a low power mode (or a power-off state) when the standby time σT of the RX circuit is not set and allowing the TX circuit to be in the low power mode (or the power-off state) and then applying the operating power to the RX circuit when the standby time σT is set; and
receiving and analyzing, by a main circuit portion of the ultrasonic diagnostic apparatus, the echo signal to generate and transmit the ultrasonic image to a user screen.

2. The method of claim 1, wherein the applying operating power to the RX circuit and then allowing the TX circuit to be in the low power mode (or the power-off state) when the standby time σT of the RX circuit is not set and allowing the TX circuit to be in the low power mode (or the power-off state) and then applying the operating power to the RX circuit when the standby time σT is set comprises:
applying the operation power to the RX circuit before a wake-up time ΔT2 necessary for driving the RX circuit to receive the ultrasonic echo signal and allowing the TX circuit for transmitting the ultrasonic signal to be in the low power mode (or the power-off state) when the standby time σT is not set (σT=0); and
allowing the TX circuit for transmitting the ultrasonic signal to be in the low power mode (or the power-off state), standing by while additionally applying the standby time σT to the wake-up time ΔT2 necessary for driving the RX circuit to receive the ultrasonic echo signal, and applying the operating power to the RX circuit when the standby time σT is set.

3. A portable ultrasonic diagnostic apparatus, to which the method of performing the low power mode according to claim 1 is to be applied, comprising:
an ultrasonic probe which comprises a piezoelectric element array module and a multiplexer (MUX) circuit portion to generate an ultrasonic wave and receive an echo signal;
a main circuit portion which receives and analyzes the echo signal received from the ultrasonic probe to generate and transmit an ultrasonic image to a user screen;
a portable battery which supplies power necessary for the ultrasonic probe and the main circuit portion; and
a low power mode controller which receives power from the portable battery to have a high voltage which drives the ultrasonic probe and generates and distributes a voltage necessary for the entire system,
wherein the main circuit portion comprises a transceiver which performs as a switch connecting one of a TX circuit for transmitting the ultrasonic wave and an RX circuit for receiving an ultrasonic echo to the ultrasonic probe depending on a transmitting and receiving state, and
wherein the low power mode controller, by controlling the transceiver, minimizes a power consumption amount by stopping an operation of the RX circuit which receives an ultrasonic echo signal reflected by a test subject when the ultrasonic signal is transmitted and stopping an operation of the TX circuit which transmits the ultrasonic signal when the ultrasonic echo signal is received.

4. The portable ultrasonic diagnostic apparatus of claim 3, wherein the main circuit portion comprises:
a high voltage generator which generates an electric pulse applied to the piezoelectric element array module to generate the ultrasonic wave;
an AD signal processor which amplifies a level of the ultrasonic echo signal returning from the test subject and converts the amplified ultrasonic echo signal into a digital signal;
the transceiver which transmits a high voltage pulse generated by the high voltage pulse generator to the ultrasonic probe and transmits an analog signal received from the ultrasonic probe to the AD signal processor;
a beam former which allows the high voltage pulse generator to generate an adequate high voltage pulse using a parameter adequate to the ultrasonic probe and receives the digital signal from the AD signal processor to perform data conversion to be appropriate for the ultrasonic probe;
a processor which allows the beam former to perform beam forming adequate to the ultrasonic probe, generates the ultrasonic image using data received from the beam former, transmits the ultrasonic image to a display portion and an external display apparatus using ultrasonic scan data, and controls the entire system; and
a communication portion which transmits and receives data with the external display apparatus.

5. The portable ultrasonic diagnostic apparatus of claim 4, wherein the communication portion uses any one of a local area network (LAN) using a cable, Bluetooth, a wireless universal serial bus (USB), a wireless LAN, wireless fidelity (Wi-Fi), Zigbee, and infrared data association (IrDA).

6. The portable ultrasonic diagnostic apparatus of claim 4, wherein the external display apparatus comprises a data communication portion which transmits and receives data with the communication portion, a menu input portion which receives a menu signal from a user, a screen display portion which displays the ultrasonic image and a menu, and a controller which transmits and receives a control signal with the processor.

7. The portable ultrasonic diagnostic apparatus of claim 6, wherein the data communication portion receives scan data from the portable ultrasonic diagnostic apparatus and transmits the scan data to the controller, the controller performs a scan conversion process of forming the ultrasonic image using the scan data and then performs post processing necessary for improving image quality, the controller performs a decompression process when the scan data sent from the portable ultrasonic diagnostic apparatus is compressed, the screen display portion displays the ultrasonic image formed by the controller on a screen to allow the user to see it, the menu input portion receives and transmits a user input to the controller, and the controller directly processes the user input or transmits the user input to the portable ultrasonic diagnostic apparatus using the data communication portion.

8. The portable ultrasonic diagnostic apparatus of claim 3, wherein the low power mode controller allows the high voltage pulse generator to operate on a preset frequency in an operation time of the TX circuit for receiving the voltage from the battery and transmitting an ultrasonic pulse and allows the AD signal processor provided in the main circuit portion to amplify and then convert the ultrasonic echo signal into the digital signal in an operation time of the RX circuit which receives the ultrasonic echo.

9. A portable ultrasonic diagnostic apparatus, to which the method of performing the low power mode according to claim 2 is to be applied, comprising:
an ultrasonic probe which comprises a piezoelectric element array module and a multiplexer (MUX) circuit portion to generate an ultrasonic wave and receive an echo signal;
a main circuit portion which receives and analyzes the echo signal received from the ultrasonic probe to generate and transmit an ultrasonic image to a user screen;
a portable battery which supplies power necessary for the ultrasonic probe and the main circuit portion; and
a low power mode controller which receives power from the portable battery to have a high voltage which drives the ultrasonic probe and generates and distributes a voltage necessary for the entire system,
wherein the main circuit portion comprises a transceiver which performs as a switch connecting one of a TX circuit for transmitting the ultrasonic wave and an RX circuit for receiving an ultrasonic echo to the ultrasonic probe depending on a transmitting and receiving state, and
wherein the low power mode controller, by controlling the transceiver, minimizes a power consumption amount by stopping an operation of the RX circuit which receives an ultrasonic echo signal reflected by a test subject when the ultrasonic signal is transmitted and stopping an operation of the TX circuit which transmits the ultrasonic signal when the ultrasonic echo signal is received.

10. The portable ultrasonic diagnostic apparatus of claim 10, wherein the main circuit portion comprises:
a high voltage generator which generates an electric pulse applied to the piezoelectric element array module to generate the ultrasonic wave;
an AD signal processor which amplifies a level of the ultrasonic echo signal returning from the test subject and converts the amplified ultrasonic echo signal into a digital signal;
the transceiver which transmits a high voltage pulse generated by the high voltage pulse generator to the ultrasonic probe and transmits an analog signal received from the ultrasonic probe to the AD signal processor;
a beam former which allows the high voltage pulse generator to generate an adequate high voltage pulse using a parameter adequate to the ultrasonic probe and receives the digital signal from the AD signal processor to perform data conversion to be appropriate for the ultrasonic probe;
a processor which allows the beam former to perform beam forming adequate to the ultrasonic probe, generates the ultrasonic image using data received from the beam former, transmits the ultrasonic image to a display portion and an external display apparatus using ultrasonic scan data, and controls the entire system; and
a communication portion which transmits and receives data with the external display apparatus.

11. The portable ultrasonic diagnostic apparatus of claim 11, wherein the communication portion uses any one of a local area network (LAN) using a cable, Bluetooth, a wireless universal serial bus (USB), a wireless LAN, wireless fidelity (Wi-Fi), Zigbee, and infrared data association (IrDA).

12. The portable ultrasonic diagnostic apparatus of claim 11, wherein the external display apparatus comprises a data communication portion which transmits and receives data with the communication portion, a menu input portion which receives a menu signal from a user, a screen display portion which displays the ultrasonic image and a menu, and a controller which transmits and receives a control signal with the processor.

13. The portable ultrasonic diagnostic apparatus of claim 12, wherein the data communication portion receives scan data from the portable ultrasonic diagnostic apparatus and transmits the scan data to the controller, the controller performs a scan conversion process of forming the ultrasonic image using the scan data and then performs post processing necessary for improving image quality, the controller performs a decompression process when the scan data sent from the portable ultrasonic diagnostic apparatus is compressed, the screen display portion displays the ultrasonic image formed by the controller on a screen to allow the user to see it, the menu input portion receives and transmits a user input to the controller, and the controller directly processes the user input or transmits the user input to the portable ultrasonic diagnostic apparatus using the data communication portion.

14. The portable ultrasonic diagnostic apparatus of claim 3, wherein the low power mode controller allows the high voltage pulse generator to operate on a preset frequency in an operation time of the TX circuit for receiving the voltage from the battery and transmitting an ultrasonic pulse and allows the AD signal processor provided in the main circuit portion to amplify and then convert the ultrasonic echo signal into the digital signal in an operation time of the RX circuit which receives the ultrasonic echo.
